(19) United States
(12) Patent Application Publication
(10) Pub. No.: US 2023/0219082 A1
(43) Pub. Date: Jul. 13, 2023

(54) MICROFLUIDIC DEVICES, SYSTEMS, AND METHODS

(71) Applicant: INTERFACE FLUIDICS LTD., CALGARY (CA)

(72) Inventors: SOURABH AHITAN, EDMONTON (CA); SHAWN WORTHING, EDMONTON (CA); ALI ABEDINI, EDMONTON (CA); TAO YANG, STAVANGER (NO); FRODE UNGAR, SANDNES (NO); KNUT ULEBERG, SANDNES (NO)

(73) Assignee: INTERFACE FLUIDICS LTD., CALGARY, AB (CA)

(21) Appl. No.: 17/997,665

(22) PCT Filed: Jun. 7, 2021
(86) PCT No.: PCT/CA2021/050778

§ 371 Date: Nov. 1, 2022

Related U.S. Application Data
(60) Provisional application No. 63/039,144, filed on Jun. 15, 2020.

(51) Int. Cl. B01L 3/00; G01N 13/00; G01N 33/28

(52) U.S. Cl. CPC B01L 3/502707; B01L 3/502715; G01N 13/00; B01L 3/50273; B01L 3/50851; G01N 33/28; B01L 2200/147; B01L 2300/0816; B01L 2400/0487; B01L 2300/0861; B01L 2200/027; B01L 2300/049

(57) ABSTRACT

A microfluidic device includes a microfluidic substrate having a porous media channel, an oil inlet port in fluid communication with the porous media channel, a fluid inlet port in fluid communication with the porous media channel, and an outlet port in fluid communication with the porous media channel. The porous media channel has a plurality of dividers that provide the porous media channel with a network of fluid pathways. A method for assessing miscibility of an oil composition and a fluid includes flowing an aliquot of a fluid through a porous media channel to displace at least an oil composition from the porous media channel, and conducting an optical investigation of the porous media channel to assess the miscibility of the oil composition and the fluid at the test pressure and test temperature.

### CROSS-REFERENCES TO RELATED APPLICATIONS

This application claims priority to U.S. Provisional Patent Application No. 63/039,144 filed on Jun. 15, 2020, which is incorporated herein by reference in its entirety.

### FIELD

This document relates to microfluidics. More specifically, this document relates to microfluidic devices such as microfluidic chips, systems including microfluidic devices, and methods for operating microfluidic devices and systems.

### BACKGROUND

U.S. Pat. No. 8,340,913 (Mostowfi et al.) discloses methods and related systems for analyzing phase properties in a microfluidic device. A fluid is introduced under pressure into a microchannel, and phase states of the fluid are optically detected at a number of locations along the microchannel. Gas and liquid phases of the fluid are distinguished based on a plurality of digital images of the fluid in the microchannel. Bi-level images can be generated based on the digital images, and the fraction of liquid or gas in the fluid can be estimated versus pressure based on the bi-level images. Properties such as bubble point values and/or a phase volume distribution ratio versus pressure for the fluid are estimated based on the detected phase states of the fluid.

### SUMMARY

The following summary is intended to introduce the reader to various aspects of the detailed description, but not to define or delimit any invention.

Methods for assessing miscibility of an oil composition and a fluid are disclosed. According to some aspects, a method for assessing miscibility of an oil composition and a fluid includes: a. in a microfluidic device, heating or cooling a porous media channel to a test temperature; b. while applying back-pressure to the porous media channel, loading the porous media channel with an aliquot of the oil composition; c. while applying back-pressure to the porous media channel to maintain the porous media channel at a test pressure, flowing an aliquot of the fluid through the porous media channel to displace at least some of the aliquot of the oil composition from the porous media channel; and d. during and/or after step c., conducting an optical investigation of the porous media channel to assess the miscibility of the oil composition and the fluid at the test pressure and the test temperature.

In some examples, in step b., back-pressure is applied to maintain the porous media channel at the test pressure.

In some examples, steps b. to d. are carried out over less than 30 minutes.

In some examples, after step c., the method further includes: e. while applying back-pressure to the porous media channel, loading the porous media channel with a subsequent aliquot of the oil composition; f. while applying back-pressure to the porous media channel to maintain the porous media channel at a subsequent test pressure, flowing a subsequent aliquot of the fluid through the porous media channel to displace at least some of the subsequent aliquot of the oil composition from the porous media channel; and g. during and/or after step f, conducting an optical investigation of the porous media channel to assess the miscibility of the oil composition and the fluid at the subsequent test pressure. The method can further include serially repeating steps e. to g. with further subsequent test pressures to ascertain the multiple contact minimum miscibility pressure of the oil composition and the fluid.

In some examples, after step c., the method further includes: e. while applying back-pressure to the porous media channel, loading the porous media channel with a subsequent aliquot of the oil composition; f. while applying back-pressure to the porous media channel to maintain the porous media channel at the test pressure, flowing a subsequent aliquot of the fluid through the porous media channel to displace at least some of the subsequent aliquot of the oil composition from the porous media channel, wherein the aliquot of the fluid has a first fluid concentration, and the subsequent aliquot of the fluid has a second fluid concentration; and g. during and/or after step f, conducting an optical investigation of the porous media channel to assess the miscibility of the oil composition and the fluid at the second fluid concentration. The method can further include serially repeating steps e. to g. with further subsequent aliquots of the fluid having further fluid concentrations to ascertain the multiple contact minimum miscibility concentration of the oil composition and the fluid.

In some examples, step d. includes detecting the presence or absence of an interface between the fluid and the oil composition at a fluid-oil displacement front. The presence of an interface can indicate that multiple contact miscibility has not been achieved between the oil composition and the fluid. The absence of an interface can indicate that multiple contact miscibility has been achieved between the oil composition and the fluid. In some examples, step d. includes assessing the oil composition displacement efficiency. An oil composition displacement efficiency of less than about 100 percent can indicate that multiple contact miscibility has not been achieved between the oil composition and the fluid, and an oil composition displacement efficiency of about 100 percent can indicate that multiple contact miscibility has been achieved between the oil composition and the fluid

In some examples, step d. is at least partially automated.

In some examples, the porous media channel has a porous media channel length of between about 25 cm and about 75 cm and a porous media channel width of between about 5 microns and about 500 microns. In some examples, the porous media channel includes a network of fluid pathways, and each fluid pathway has a pathway width of between about 1 micron and about 50 microns.

In some examples, the fluid is a gas, a liquid, and/or a supercritical fluid. In some examples, the oil composition is at least one of a live oil, a dead oil, a gas, a liquid, a supercritical composition, a single-component composition, and a multi-component composition.

In some examples, step c. includes flowing the aliquot of the fluid into the porous media channel via a fluid inlet channel. The porous media channel can have a porous media channel cross-sectional area, and the fluid inlet channel can have a fluid inlet channel cross-sectional area that is less than the porous media channel cross-sectional area.

In some examples, prior to loading the porous media channel with the aliquot of the oil composition, the method can further include flashing the aliquot of the oil composition into a liquid phase and a gas phase in a flash zone of the microfluidic device. Flashing the aliquot of the oil composition into the liquid phase and the gas phase in the flash zone of the microfluidic device can include flowing the aliquot of the oil composition into the porous media channel via an oil inlet channel and a feeder channel downstream of the oil inlet channel. The oil inlet channel can have an oil inlet channel cross-sectional area, and the feeder channel can have a feeder channel cross-sectional area that is greater than the oil inlet channel cross-sectional area.

In some examples, the method further includes passing the aliquot of the oil composition through a filter zone of the microfluidic device to filter the aliquot of the oil composition prior to loading the aliquot of the oil composition into the porous media channel. Step b. can include flowing the aliquot of the oil composition into the porous media channel via a first oil inlet channel and a network of secondary oil inlet channels. The first oil inlet channel and the network of secondary oil inlet channels can form the filter zone. The first oil inlet channel can have a first cross-sectional area, the secondary oil inlet channels can have a second cross-sectional area, and the second cross-sectional area can be less than the first cross-sectional area.

Microfluidic systems are also disclosed. According to some aspects, a microfluidic system includes a microfluidic device, an oil injection sub-system, a fluid injection sub-system, a pressure regulation sub-system, a manifold, a temperature regulation sub-system, and an optical investigation sub-system. The microfluidic device includes a microfluidic substrate, and the microfluidic substrate has a porous media channel, an oil inlet port in fluid communication with the porous media channel, a fluid inlet port in fluid communication with the porous media channel, and an outlet port in fluid communication with the porous media channel. The porous media channel includes a plurality of dividers that provide the porous media channel with a network of fluid pathways. The oil injection sub-system is in fluid communication with the oil inlet port for forcing an oil composition into the network of fluid pathways. The fluid injection sub-system is in fluid communication with the fluid inlet port for forcing a fluid through the network of fluid pathways from the fluid inlet port towards the outlet port. The pressure regulation sub-system regulates the pressure in the network of fluid pathways. The manifold provides fluid communication between the microfluidic substrate and the oil injection sub-system, the fluid injection sub-system, and the pressure regulation sub-system. The temperature regulation sub-system regulates the temperature of at least the microfluidic device. The optical investigation sub-system can optically access at least a portion of the porous media channel.

In some examples, the pressure regulation sub-system includes a backpressure regulator in fluid communication with the outlet port.

In some examples, the system further includes a control sub-system connected to the oil injection sub-system, the fluid injection sub-system, the pressure regulation sub-system, the temperature regulation sub-system, and the optical investigation sub-system, for providing automatic control of the microfluidic system.

In some examples, the porous media channel is serpentine. In some examples, the porous media channel has a porous media channel length of between about 25 cm and about 75 cm and a porous media channel width of between about 5 microns and about 500 microns. In some examples, each fluid pathway has a pathway width of between about 1 micron and about 50 microns.

In some examples, the dividers are in the form of posts that are created by etching the fluid pathways into the substrate. The posts can be positioned in an array. The posts can be randomly positioned.

In some examples, the microfluidic substrate further includes an oil inlet channel extending towards the porous media channel from the oil inlet port, an outlet channel extending towards the porous media channel from the outlet port, and a fluid inlet channel extending towards the porous media channel from the fluid inlet port.

In some examples, the microfluidic substrate further includes at least a first feeder channel, and the oil inlet channel is in fluid communication with the porous media channel via the first feeder channel. The oil inlet channel can have an oil inlet channel cross-sectional area, and the first feeder channel can have a first feeder channel cross-sectional area that is greater than the oil inlet channel cross-sectional area, to form a flash zone of the microfluidic device.

In some examples, the porous media channel has a porous media channel depth, the fluid inlet channel has a fluid inlet channel depth, and the fluid inlet channel depth is less than the porous media channel depth.

In some examples, the microfluidic substrate further includes a secondary oil inlet port. The oil inlet port and the secondary oil inlet port can be in fluid communication with each other via a first oil inlet channel. The microfluidic substrate can further include a network of secondary oil inlet channels. The first oil inlet channel can be in fluid communication with the porous media channel via the network of secondary oil inlet channels. In some examples, the first oil inlet channel has a first cross-sectional area, the secondary oil inlet channels each have a second cross-sectional area, and the second cross-sectional area is less than the first cross-sectional area to form a filter zone in the microfluidic device.

Microfluidic devices are also disclosed. According to some aspects, a microfluidic device includes a microfluidic substrate having a porous media channel, an oil inlet port in fluid communication with the porous media channel, a fluid inlet port in fluid communication with the porous media channel, and an outlet port in fluid communication with the porous media channel. The porous media channel has a plurality of dividers that provide the porous media channel with a network of fluid pathways.

In some examples, the porous media channel has a porous media channel length and a porous media channel width, and a ratio of the porous media channel length to the porous media channel width is at least 1000:1.

In some examples, the porous media channel is serpentine.

In some examples, the porous media channel has a porous media channel length of at least about 1 cm. In some examples, the porous media channel has a porous media channel length of at least about 5 cm. In some examples, the porous media channel has a porous media channel length of between about 5 cm and about 400 cm. In some examples, the porous media channel has a porous media channel length of between about 25 cm and about 75 cm.

In some examples, the porous media channel has a porous media channel width of at least about 5 microns. In some examples, the porous media channel has a porous media channel width of between about 5 microns and about 500 microns. In some examples, the porous media channel has a porous media channel width of between about 50 microns and 300 microns.

In some examples, the fluid pathways have a pathway width of at least about 1 micron. In some examples, the fluid pathways have a pathway width of between about 1 micron and about 50 microns. In some examples, the fluid pathways have a pathway width of between about 2 microns and about 20 microns.

In some examples, the dividers are in the form of posts that are created by etching the fluid pathways into the substrate. The posts can be positioned in an array. The posts can be positioned randomly.

In some examples, the microfluidic device further includes an oil inlet channel extending towards the porous media channel from the oil inlet port, an outlet channel extending towards the porous media channel from the outlet port, and a fluid inlet channel extending towards the porous media channel from the fluid inlet port.

In some examples, the microfluidic device further includes at least a first feeder channel, and the oil inlet channel is in fluid communication with the porous media channel via the first feeder channel. In some examples, the oil inlet channel has an oil inlet channel cross-sectional area, and the first feeder channel has a first feeder channel cross-sectional area that is greater than the oil inlet channel cross-sectional area, to form a flash zone of the microfluidic device.

In some examples, the porous media channel has a porous media channel depth, the fluid inlet channel has a fluid inlet channel depth, and the fluid inlet channel depth is less than the porous media channel depth. In some examples, the fluid inlet channel depth is at least 10 times less than the porous media channel depth. In some examples, the fluid inlet channel depth is between about 25 times less and 75 times less than the porous media channel depth.

In some examples, the microfluidic device further includes a secondary oil inlet port. The oil inlet port and the secondary oil inlet port can be in fluid communication with each other via a first oil inlet channel. The microfluidic device can further include a network of secondary oil inlet channels. The first oil inlet channel can be in fluid communication with the porous media channel via the network of secondary oil inlet channels. In some examples, the first oil inlet channel has a first cross-sectional area, the secondary oil inlet channels each have a second cross-sectional area, and the second cross-sectional area is less than the first cross-sectional area to form a filter zone in the microfluidic substrate.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a perspective view of an example microfluidic device;

FIG. 2A is a plan view of the microfluidic device of FIG. 1;

FIG. 2B is an enlarged view of a portion of FIG. 2A;

FIG. 3 is a schematic view of an example microfluidic system including the microfluidic device of FIGS. 1 to 2B;

FIG. 4A is a still image captured using the system of FIG. 3, showing that multiple contact miscibility between an oil composition and a fluid has not been achieved;

FIG. 4B is another still image captured using the system of FIG. 3, showing that multiple contact miscibility between an oil composition and a fluid has been achieved;

FIG. 5 is a plan view of another example microfluidic device;

FIG. 6 is a plan view of another example microfluidic device;

FIG. 7A is a plan view of another example microfluidic device;

FIG. 7B is an enlarged view of a portion of FIG. 7A;

FIG. 8 is a plan view of another example microfluidic device;

FIG. 9 is a plan view of another example microfluidic device;

FIG. 10 is a plan view of another example microfluidic device;

FIG. 11 is a plan view of another example microfluidic device;

FIG. 12 is a plan view of another example microfluidic device; and

FIG. 13 is a plan view of another example microfluidic device.

### DETAILED DESCRIPTION

Various apparatuses or processes or compositions will be described below to provide an example of an embodiment of the claimed subject matter. No embodiment described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are microfluidic devices in the form of microfluidic chips, systems incorporating microfluidic devices, and related methods. The microfluidic devices, systems, and methods can be used, for example, in the oil and gas industry, in order to predict behavior of fluids and oil compositions in oil-bearing porous subterranean formations (e.g. in shale and/or tight oil formations, as well as fracture zones (also known as “frac zones”) created in such formations during hydraulic fracturing). More specifically, the microfluidic devices, systems, and methods can be used, for example, in order to assess miscibility of fluids and oil compositions. For example, the microfluidic devices, systems, and methods disclosed herein can be used to assess the multiple contact minimum miscibility pressure (MMP) of a crude oil and a solvent, and/or the multiple contact minimum miscibility concentration (MMC) of a crude oil and a solvent, in order to predict the behavior of the crude oil and the solvent in an oil-bearing porous subterranean formation.

As used herein, the term “assess” includes (but is not limited to) precise determination, estimation, prediction, analysis, testing, and study. For example, the statement that “microfluidic devices can be used to assess the multiple contact MMP of a crude oil and a solvent” indicates that microfluidic devices can be used to precisely determine, to estimate, to predict, to analyze, to test, and/or to study the multiple contact MMP of a crude oil and a solvent.

As used herein, the term “oil composition” refers to a composition that includes or is made up of an oil. An oil composition may be synthetic or naturally derived. An oil composition can be a crude oil, or a crude oil fraction (e.g. a portion of a crude oil that has been distilled or otherwise separated from the crude oil). An oil composition can be a sample that resembles (e.g. has a composition substantially similar to) a crude oil or a crude oil fraction. An oil composition can be a dead oil (i.e. an oil composition taken from a subterranean formation and that does not flash at ambient temperature and pressure) or a live oil (i.e. an oil composition taken from a subterranean formation and having dissolved gases that spontaneously evolve at ambient pressure and temperature). An oil composition can be a gas, a liquid, and/or a supercritical composition. An oil composition can be a single-component composition or a multi-component composition.

As used herein, the term “fluid” refers to any fluid that can be mixed with an oil composition. The term “fluid” can refer to a liquid, a gas, a supercritical fluid, or a combination thereof. The term “fluid” can refer to a single-component fluid, or a mixture of different components. “Solvents” used in the oil and gas industry are examples of fluids. Such solvents can include, for example, carbon dioxide, nitrogen, methane, ethane, propane, hydrogen sulfide, n-butane, iso-butane, natural gas, natural gas liquids, and produced gas (e.g., gas produced by a subterranean formation).

In general, the microfluidic device, systems, and methods disclosed herein can allow for fast, accurate, and reliable assessment of parameters such as multiple contact MMP, and multiple contact MMC. For example, the multiple contact MMP of a given solvent and a given crude oil can be assessed in a matter of hours (e.g. up to twelve hours). Furthermore, the systems and methods disclosed herein can be automated and precisely controlled, which can allow for accuracy as well as reduced costs and reduced manpower.

In general, the microfluidic devices disclosed herein can include a channel, referred to herein as a “porous media channel” (as described in further detail below), that enables multiple contact between an oil composition and a fluid, to facilitate multiple contact miscibility. The porous media channel can be loaded with an oil composition (e.g. a sample of live oil from a porous subterranean formation), and a fluid (e.g. a solvent such as carbon dioxide) can then be forced through the porous media channel, to displace at least some of the oil composition. As the fluid is forced through the porous media channel (or after the flow of fluid through the porous media channel has stopped), an optical investigation of the porous media channel can be conducted (for example with the use of a microscope, and either in real time or by analyzing a video recording or still images) to assess the miscibility of the fluid and the oil composition. For example, the porous media channel can be heated or cooled to a test temperature, and loaded with the oil composition while applying back-pressure to pressurize the porous media channel to a test pressure. An optical investigation can then be conducted as the fluid is forced through the porous media channel at the test pressure and the test temperature, to assess the miscibility of the fluid and the oil composition at the test pressure and the test temperature (e.g. to determine the multiple contact MMP of the oil composition and the fluid). The optical investigation can include, for example, visual inspection to determine whether an interface exists between the fluid and the oil composition in the porous media channel. If an interface is present, and more specifically, if an interface is present for the duration of the flow of fluid through the porous media channel (i.e. even at the minimum miscibility pressure, an interface may initially exist between the fluid and the oil composition at the fluid-oil displacement front; however, the interface will disappear as the fluid-oil displacement front moves along the length of the porous media channel), it can be concluded that multiple contact miscibility of the fluid and the oil composition has not occurred at the test pressure and the test temperature. If an interface is not present (i.e. if an interface is never present or if an interface is initially present but disappears), it can be concluded that multiple contact miscibility of the fluid and the oil composition has occurred at the test pressure and the test temperature. Alternatively or in addition to determining whether an interface exists between the fluid and the oil composition, the optical investigation can include, for example, determining the oil composition displacement efficiency. This process can optionally be repeated with subsequent test pressures (optionally at the same test temperature) until multiple contact miscibility is achieved and the multiple contact MMP of the oil composition and the fluid is determined (i.e. until the visual inspection determines that an interface is not present at a given test pressure, or until the displacement efficiency indicates that the oil composition and the fluid are miscible). Similarly, this process can optionally be repeated with subsequent fluid concentrations (optionally at the sample pressure and temperature) until multiple contact miscibility is achieved and the multiple contact MMC of the oil composition and the fluid is determined (i.e. until the visual inspection determines that an interface is not present at a given test pressure, or until the displacement efficiency indicates that the oil composition and the fluid are miscible).

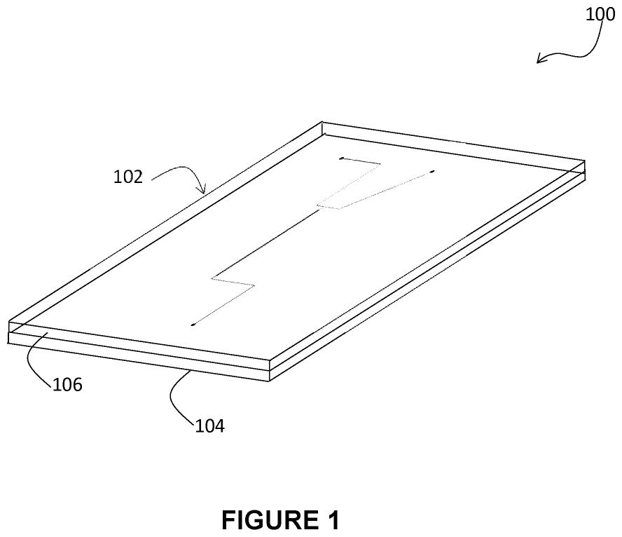

Referring now to FIG. 1, an example microfluidic device 100 is shown. The microfluidic device 100 may also be referred to as a “microfluidic chip”. The microfluidic device 100 includes a microfluidic substrate 102 that has various microfluidic features therein (i.e. fluid channels, fluid pathways, dividers, and fluid ports, described in further detail below). The microfluidic substrate 102 allows for optical investigation (e.g. imaging, optionally with the use of an optical microscope and/or video recording equipment and/or a photographic camera) of at least some of the microfluidic features.

Referring still to FIG. 1, in the example shown, the substrate 102 includes a base panel 104 in which the microfluidic features are etched, and a cover panel 106 that is secured to the base panel 104 and that covers the microfluidic features. In the example shown, the base panel 104 is an opaque silicon panel, and the cover panel 106 is a transparent glass panel. In alternative examples, the substrate 102 may be of another configuration. For example, both the base panel 104 and the cover panel 106 can be a transparent glass panel, or the base panel 104 can be a transparent glass panel while the cover panel 106 can be an opaque silicon panel.

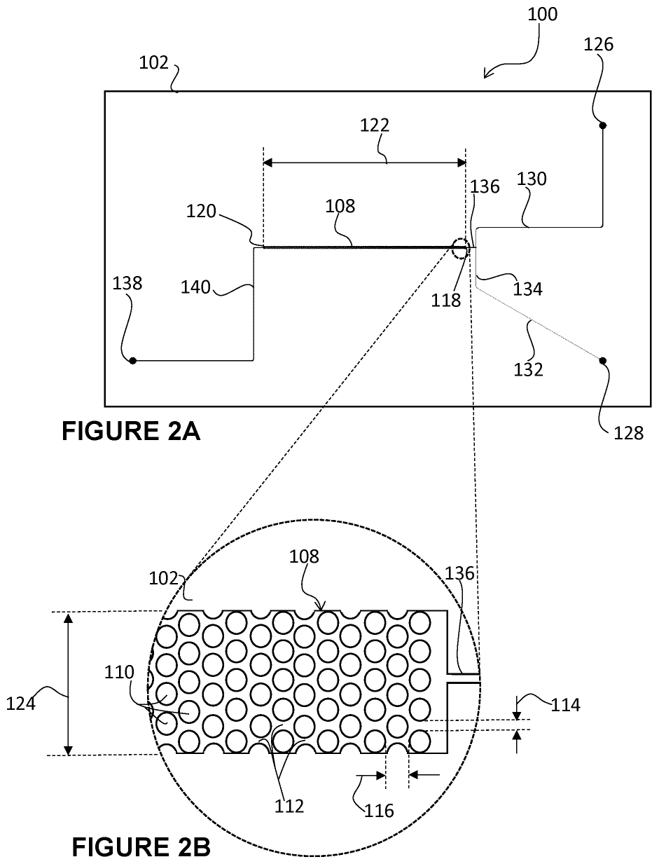

Referring now to FIGS. 2A and 2B, the substrate 102 includes a porous media channel 108, as mentioned above. As used herein, the term “channel” refers to a narrow and elongate (e.g. having a length that is greater than its width, such as a length to width ratio of at least 10:1 or at least 25:1 or at least 50:1 or at least 100:1) feature through which substances (e.g. fluids and/or oil compositions) can flow. The term “porous media channel” refers to a channel that enables multiple contact between an oil composition and a fluid, to facilitate multiple contact miscibility. Referring to FIG. 2B, in the example shown, in order to enable multiple contact between an oil composition and a fluid, the porous media channel 108 includes a plurality of dividers 110 (only some of which are labelled), which are in the form of posts. The dividers 110 provide the porous media channel 108 with a network of fluid pathways 112 (only some of which are labelled), which enable multiple contact between an oil composition and a fluid. The posts can be created, for example, by etching the fluid pathways 112 into the base panel 104, leaving the posts as unetched sections.

Referring still to FIG. 2B, in the example shown, the posts are generally circular in cross-section, have a diameter of about 32 microns, and are positioned in a regular array. In alternative examples (some of which are described below), the posts can be of various other configurations (i.e. any configuration that enables multiple contact). For example, the posts can be hexagonal or square or random shapes in cross-section, can have a diameter of between about 4 microns and about 64 microns, and/or can be positioned in another configuration (such as at random).

Referring still to FIG. 2B, in the example shown, the fluid pathways 112 each have a width 114 (also referred to herein as a “pathway width”) of about 8 microns. In alternative examples (some of which are described in further detail below), the pathway width can be of another size, such at least 1 micron, or between about 1 micron and about 50 microns, or between about 2 microns and about 20 microns. Furthermore, in alternative examples (such as those where the posts are positioned at random), each pathway width can be different, or some of the pathway widths can be different from others. The fluid pathways further have a length 116 (also referred to herein as a “pathway length”), which can be the same as the pathway width, or different from the pathway width.

Referring back to FIG. 2A, in the example shown, the porous media channel 108 has a first end 118 and a second end 120, and a length 122 (also referred to herein as a “porous media channel length”) that is defined between the first end 118 and the second end 120. In the example shown, the length 122 is about 1.4 cm. Referring to FIG. 2B, the porous media channel 108 further has a width 124 (also referred to herein as a “porous media channel width”). In the example shown, the porous media channel width 124 is about 200 microns. Furthermore, the porous media channel has a depth (also referred to herein as a “porous media channel depth”). In the example shown, the porous media channel depth is about 50 microns.

In alternative examples (some of which are described below), the porous media channel can be of various other lengths, widths, and depths. For example, the porous media channel length can be at least about 1 cm, or at least about 5 cm, or between about 5 cm and about 400 cm, or between about 25 cm and about 75 cm. Furthermore, the porous media channel width can be, for example, at least about 5 microns, or between about 5 microns and about 500 microns, or between about 5 microns and about 300 microns. Furthermore, the porous media channel depth can be, for example, between about 0.001 microns and about 100 microns (e.g. about 0.1 microns, or about 0.05 microns, or about 50 microns). Although the porous media channel can have wide range of lengths, relatively large lengths (e.g. where the ratio of the porous media channel length to the porous media channel width is at least 1000:1) may more reliably facilitate multiple contact miscibility.

Referring to FIG. 2A, in the example shown, the porous media channel is of a straight configuration (i.e. it extends linearly between the first end 118 and the second end 120). In alternative examples (some of which are described below), particularly those in which the porous media channel length is relatively large (i.e. larger than the length of the substrate itself), the porous media channel can be of a non-straight configuration, in order to accommodate its length within the area of the substrate. For example, the porous media channel can be serpentine (as described in further detail below).

Referring still to FIG. 2A, the microfluidic substrate 102 further includes an oil inlet port 126, and a fluid inlet port 128, each of which is in fluid communication with the porous media channel 108. Particularly, in the example shown, the oil inlet port 126 is in fluid communication with the first end 118 of the porous media channel 108 via an oil inlet channel 130 that extends towards the porous media channel 108 from the oil inlet port 126, for loading an oil composition into the porous media channel 108. The fluid inlet port 128 is in fluid communication with the first end 118 of the porous media channel 108 via a fluid inlet channel 132 that extends towards the porous media channel 108 from the fluid inlet port 128, for loading a fluid into the porous media channel 108. The microfluidic substrate 102 further includes a pair of feeder channels (i.e. a first feeder channel 134 and a second feeder channel 136). The oil inlet channel 130 and fluid inlet channel 132 are in fluid communication with the porous media channel 108 via the first feeder channel 134, which joins to the oil inlet channel 130 and the fluid inlet channel 132, and via the second feeder channel 136, which extends from the first feeder channel 134 to the porous media channel 108.

Referring still to FIG. 2A, the microfluidic substrate 102 further includes an outlet port 138, for allowing egress of the oil composition and the fluid from the porous media channel 108. In the example shown, the outlet port 138 is in fluid communication with the second end 120 of the porous media channel 108 via an outlet channel 140 that extends towards the porous media channel 108 from the outlet port 138.

The oil inlet port 126, fluid inlet port 128, oil inlet channel 130, fluid inlet channel 132, first feeder channel 134, second feeder channel 136, outlet port 138, and outlet channel 140 can be etched and/or drilled into the base panel 104 (shown in FIG. 1) of the substrate 102.

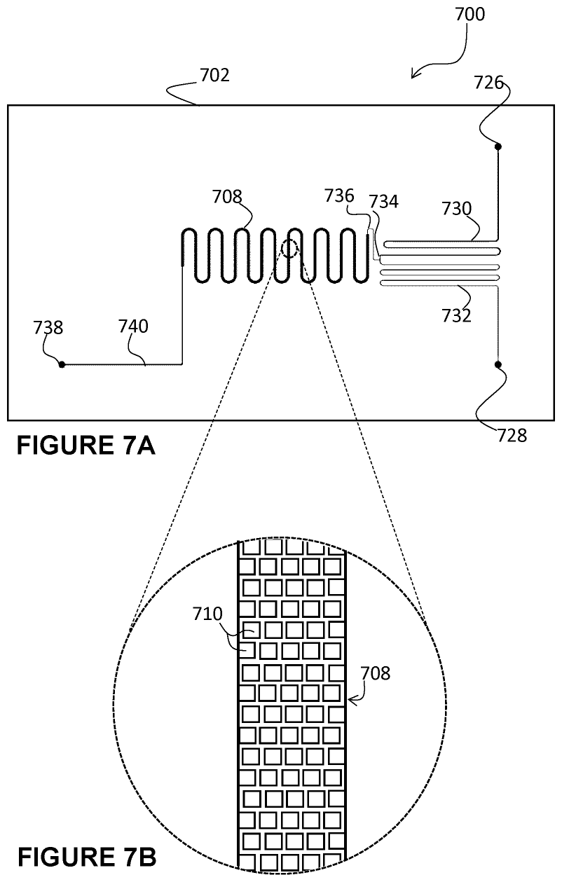

Each of the oil inlet channel 130, fluid inlet channel 132, first feeder channel 134, second feeder channel 136, and outlet channel 140 have a respective length (also referred to herein as an “oil inlet channel length”, a “fluid inlet channel length”, a “first feeder channel length”, a “second feeder channel length”, and an “outlet channel length”, respectively), a respective width (also referred to herein as an “oil inlet channel width”, a “fluid inlet channel width”, a “first feeder channel width”, a “second feeder channel width”, and an “outlet channel width”, respectively), and a respective depth (also referred to herein as an “oil inlet channel depth”, a “fluid inlet channel depth”, a “first feeder channel depth”, a “second feeder channel depth”, and “an outlet channel depth”, respectively). In the example shown, the oil inlet channel 130 and fluid inlet channel 132 each have a relatively small length; in alternative examples (e.g. as shown in FIG. 7A, described below), the oil inlet channel and/or fluid inlet channel can have a relatively large length. Larger lengths can result in a relatively large pressure drop across the length of the oil inlet channel 130 and the fluid inlet channel 132, respectively. This can help to control the flow of oil compositions and fluids (e.g. low viscosity fluids, including gases such as carbon dioxide), and can also help to dampen flow pulsations caused, for example, by temperature fluctuations.

In some examples, the oil inlet channel 130, fluid inlet channel 132, first feeder channel 134, second feeder channel 136, and outlet channel 140 are all of the same width and all of the same depth. For example, the width of each of the oil inlet channel 130, the fluid inlet channel 132, the first feeder channel 134, the second feeder channel 136, and the outlet channel 140 can be about 50 microns. For further example, the width of each of the oil inlet channel 130, the fluid inlet channel 132, the first feeder channel 134, the second feeder channel 136, and the outlet channel 140 can be about 5 microns. For further example, the depth of each of the oil inlet channel 130, the fluid inlet channel 132, the first feeder channel 134, the second feeder channel 136, and the outlet channel 140 can be about 50 microns. For further example, the depth of each of the oil inlet channel 130, the fluid inlet channel 132, the first feeder channel 134, the second feeder channel 136, and the outlet channel 140 can be about 0.1 microns.

In other examples, the oil inlet channel 130, fluid inlet channel 132, first feeder channel 134, second feeder channel 136, and outlet channel 140 may be of different depths and widths. For example, the fluid inlet channel 132 can be provided with a reduced cross-sectional area (where the phrase “cross-sectional area” refers to the area of a cross-section taken perpendicular to the direction of flow) (e.g. a reduced depth and/or a reduced width), which can result in a relatively large pressure drop across the length of the fluid inlet channel 132, which in turn can help to control the flow of fluids with a low viscosity (e.g. a gas such as carbon dioxide), and can also help to dampen flow pulsations caused, for example, by temperature fluctuations. For example, the depth and/or the width of the fluid inlet channel 132 can be less than the depth of the first feeder channel 134, second feeder channel 136, and porous media channel 108, such as at least 10 times less than the depth and/or width of the first feeder channel 134, second feeder channel 136, and porous media channel 108, or between about 25 and about 75 times less than the depth and/or width of the first feeder channel 134, second feeder channel 136, and porous media channel 108. In some particular examples, the depth of the porous media channel 108, oil inlet channel 130, first feeder channel 134, second feeder channel 136, and outlet channel 140 is about 50 microns, while the depth of the fluid inlet channel 132 is about 1 micron. In such examples, the width of the oil inlet channel 130 and the outlet channel 140 can be about 50 microns, and the width of the fluid inlet channel 132, first feeder channel 134, and second feeder channel can be about 5 microns.

The terms “oil inlet port”, “oil inlet channel”, “fluid inlet port”, “fluid inlet channel”, “feeder channel”, “outlet port”, and “outlet channel” are used herein for simplicity, and are not intended to limit the use of these ports and channels. For example, while the “oil inlet port 126” may in many examples be used to load an oil composition into the microfluidic device 100, it may in other examples be used to load other materials, or may be used for egress of materials from the microfluidic device 100.

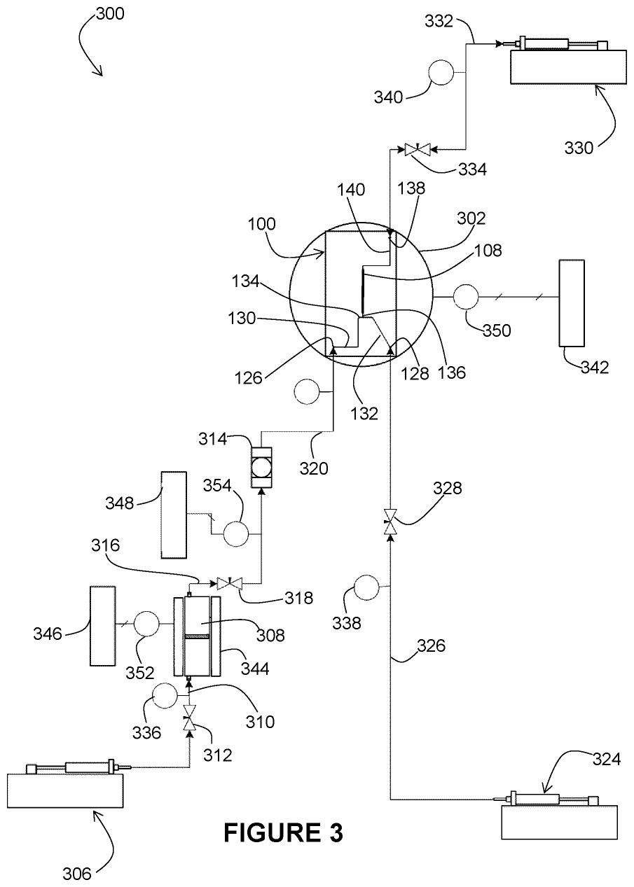

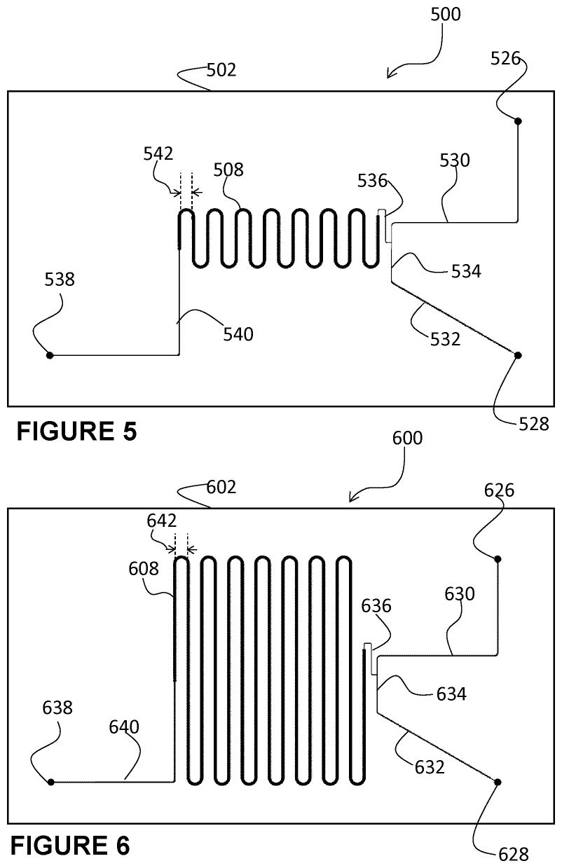

Referring now to FIG. 3, an example microfluidic system 300 is shown. As shown, the microfluidic system 300 includes the microfluidic device 100 of FIGS. 1 to 2B; however, in alternative examples, the microfluidic system 300 can include various other microfluidic devices, such as those described below with regards to FIGS. 5 to 13. Furthermore, the microfluidic device 100 can be used in various other microfluidic systems.

Referring still to FIG. 3, in the example shown, the microfluidic device 100 is supported by a manifold 302 (which can also be referred to as a “holder”), which supports the microfluidic device 100, helps to distribute pressures across the microfluidic device 100, heats or cools the microfluidic device 100, and provides for fluid communication between other parts of the system (i.e. an oil injection sub-system, a fluid injection sub-system, and a pressure regulation sub-system, as described below) and the microfluidic device 100. Examples of suitable holders are described in international patent application publication no. WO 2020/037398 (de Haas et al.) and in U.S. patent application publication no. 2020/0309285 (Sinton et al.), which are incorporated herein by reference in their entirety.

Referring still to FIG. 3, the microfluidic system 300 further includes an oil injection sub-system in fluid communication with the oil inlet port 126 of the microfluidic device 100 via the manifold 302, for forcing an oil composition into the microfluidic device 100. That is, the oil injection sub-system can force an oil composition into the network of fluid pathways 112 (shown in FIG. 2B) of the porous media channel 108, via the oil inlet port 126, the oil inlet channel 130, the first feeder channel 134, and the second feeder channel 136. In the example shown, the oil injection sub-system includes a first syringe pump 306 that is hydraulically connected to an oil storage cylinder 308 via line 310 and valve 312. The oil storage cylinder 308 can store, for example, a sample of live oil that is to be assessed with the system 300. The oil storage cylinder 308 is in fluid communication with a high-pressure filter 314 via line 316 and valve 318. The high-pressure filter 314 is in fluid communication with the oil inlet port 126 of the microfluidic device 100, via line 320 and via the manifold 302.

Referring still to FIG. 3, the microfluidic system 300 further includes a fluid injection sub-system that is in fluid communication with the fluid inlet port 128 of the microfluidic device 100 via the manifold 302, for forcing a fluid into the microfluidic device 100. The fluid injection sub-system can force a fluid through the network of fluid pathways 112 (shown in FIG. 2A) of the porous media channel 108, from the fluid inlet port 128 towards the outlet port 138 (i.e. from the fluid inlet port 128 and through the fluid inlet channel 132 to the first feeder channel 134, from the first feeder channel 134 to the second feeder channel 136, from the second feeder channel 136 through the network of fluid pathways 112 of the porous media channel 108, and from the porous media channel 108 into the outlet channel 140). In the example shown, the fluid injection sub-system includes a second syringe pump 324 that is in fluid communication with the fluid inlet port 128 of the microfluidic device 100 via line 326 and valve 328.

Referring still to FIG. 3, the microfluidic system 300 further includes a pressure regulation sub-system, for regulating the pressure within the microfluidic device 100 (i.e. for regulating the pressure within in the network of fluid pathways 112 (shown in FIG. 2B) of the porous media channel 108). In the example shown, the pressure regulation sub-system includes a backpressure regulator in the form of a third syringe pump 330, which is in fluid communication with the outlet port 138 of the microfluidic device 100 via line 332 and valve 334. The pressure regulation system further includes a pressure transducer 336 for measuring the pressure in line 310, a pressure transducer 338 for measuring the pressure in line 326, and a pressure transducer 340 for measuring the pressure in line 332.

Referring still to FIG. 3, the microfluidic system further includes a temperature regulation sub-system, for regulating the temperature of at least the microfluidic device 100 (i.e. for regulating the temperature of the network of fluid pathways 112 of the porous media channel 108). In the example shown, the temperature regulation sub-system includes a first heater 342 for regulating the temperature of the microfluidic device 100 by heating the manifold 302, a heating jacket 344 surrounding the oil storage cylinder 308 and a second heater 346 for heating the heating jacket 344, a third heater 348 for heating line 316, and temperature transducers 350, 352, and 354, respectively, connected to each of the heaters 342, 346, and 348. In alternative examples, the temperature regulation sub-system can be configured to cool microfluidic device 100 and/or other parts of the system.

The microfluidic system 300 can further include an optical investigation sub-system (not shown), for optically accessing the porous media channel 108 (i.e. the entire porous media channel 108 or a portion thereof), and optionally other features of the microfluidic device 100. The optical investigation sub-system can include, for example, one or more microscopes having a viewing window in which a portion of the porous media channel 108 can sit, one or more video cameras, and/or one or more still image cameras. The optical investigation sub-system can be computerized and can further include image processing software and image analysis software. The image processing software can optionally automatically process images captured by the optical investigation sub-system, and the image analysis software can optionally automatically analyze images the processed images.

The microfluidic system 300 can further include a control sub-system (not shown) connected to the oil injection sub-system, the fluid injection sub-system, the pressure regulation sub-system, the temperature regulation system, and the optical investigation sub-system. The control sub-system can include one or more processors, which can receive, process, and/or store information received from the oil injection sub-system, the fluid injection sub-system, the pressure regulation sub-system, the temperature regulation sub-system, and the optical investigation sub-system. For example, the control system can receive temperature information from the temperature transducers 350, 352, and 354, and pressure information from the pressure transducers 336, 338, and 340. Furthermore, the control sub-system can send instructions to the oil injection sub-system, the fluid injection sub-system, the pressure regulation sub-system, the temperature regulation system, and/or the optical investigation sub-system. For example, the control system can instruct the temperature regulation system to increase and/or decrease the output of one or more of the heaters 342, 346, and 348. The control sub-system can optionally provide automatic control of the microfluidic system 300. For example, the control sub-system can be configured to automatically instruct the temperature regulation system to increase and/or decrease the output of one or more of the heaters 342, 346, and 348, based on the received temperature information. The control sub-system can provide similar instructions to the pressure regulation system.

A method of assessing miscibility of a fluid and an oil composition will now be described. The method will be described with reference to the microfluidic device 100 and the microfluidic system 300; however, the method is not limited to the microfluidic device 100 and the microfluidic system 300, and the microfluidic device 100 and microfluidic system 300 are not limited to operation in accordance with the method. Furthermore, for clarity, the method with be described with reference to a certain sequence of steps (e.g. a given step may be described as “a first step” or “a second step”, or terms such as “then” or “next” may be used); however, unless expressly indicated as such in the claims, the method is not limited to any particular sequence of steps.

In general, the method can include heating or cooling the porous media channel 108; while applying back-pressure to the porous media channel 108, loading the porous media channel 108 with an aliquot of an oil composition; while applying back-pressure to the porous media channel 108, flowing an aliquot of a fluid through the porous media channel 108 to displace at least some of the aliquot of the oil composition from the porous media channel 108; and conducting an optical investigation of the porous media channel 108 to assess the miscibility of the oil composition and the fluid.

More specifically, referring to FIG. 3, as a first step, the temperature regulation system can be engaged, to heat the porous media channel 108 of the microfluidic device 100 to a test temperature, and also to heat the oil storage cylinder 344 and line 316. The test temperature can be, for example, between about 25 degrees C. and about 150 degrees C.

While continuing to maintain the porous media channel 108 at the test temperature, valve 334 can be opened and the third syringe pump 330 can be engaged, to apply back pressure to the porous media channel 108. While applying back pressure, valves 312 and 318 can be opened, and the first syringe pump 306 can be engaged, to force an aliquot of the oil composition from the oil storage cylinder 308 into the network of fluid pathways 112 of the porous media channel 108, to load the porous media channel 108 with the aliquot of the oil composition. While loading the oil composition into the porous media channel 108, back-pressure can be applied to pressurize the porous media channel 108 to pressure of, for example, up to about 300 bar (e.g. about 58 bar or about 70 bar or about 82.5 bar or about 97.5 bar or about 105 bar, or about 142.5 bar).

Once loading of the porous media channel 108 with the aliquot of the oil composition is complete, valves 312 and 318 can be closed and the first syringe pump 306 can be disengaged. Then, while continuing to maintain the porous media channel 108 at the test temperature and while continuing to apply back pressure to the porous media channel 108 with the third syringe pump 330, valve 328 can be opened and the second syringe pump 324 can be engaged, to force an aliquot of fluid to flow from the second syringe pump 324 into the microfluidic device 100, and through the network of fluid pathways 112 of the porous media channel 108 of the microfluidic device 100. As the aliquot of fluid flows through the network of fluid pathways 112 of the porous media channel 108, it will displace at least some of the aliquot of the oil composition from the network of fluid pathways 112 of the porous media channel 108. While flowing the fluid through the porous media channel 108, back-pressure can be applied to pressurize the network of fluid pathways 112 of the porous media channel 108 to pressure of, for example, up to about 300 bar (e.g. about 58 bar or about 70 bar or about 82.5 bar or about 97.5 bar or about 105 bar, or about 142.5 bar). The pressure that is maintained in the porous media channel 108 during this step (i.e. while flowing the aliquot of fluid through the porous media channel 108) is referred to herein as a “test pressure”. The pressure in the porous media channel 108 is preferably the same during the step of loading the porous media channel 108 with the aliquot of the oil composition and during the step of flowing the aliquot of fluid into the porous media channel 108. That is, the test pressure is preferably maintained while loading porous media channel 108 with the aliquot of the oil composition, and while flowing the aliquot of the fluid through the porous media channel 108.

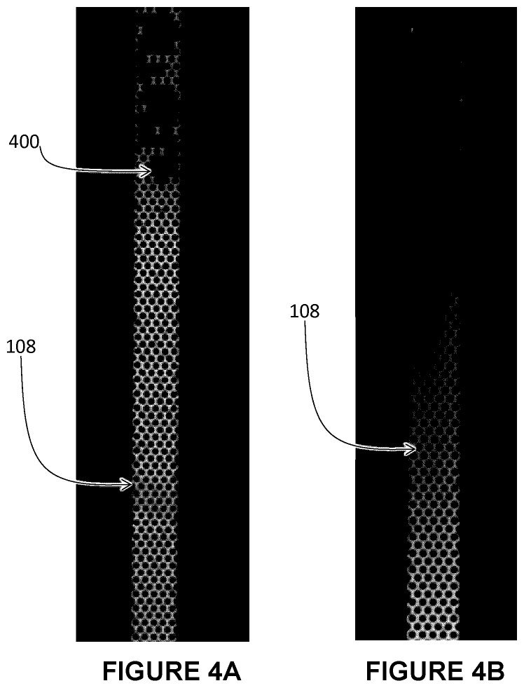

Either while the aliquot of the fluid is flowing through the porous media channel 108, or after the flow of the aliquot of the fluid through the porous media channel 108 has been stopped, an optical investigation of the porous media channel 108 can be conducted, in order to assess the miscibility of the oil composition and the fluid at the test pressure and test temperature. For example, while the aliquot of the fluid is flowing through the porous media channel 108, the optical investigation sub-system can be used to detect the presence or absence of an interface between the fluid and the oil composition at the fluid-oil displacement front. As used herein, the term “interface” refers to a visibly distinct (either by eye or by computer image analysis) boundary between the two phases. The continued presence of an interface (i.e. until the displacement front leaves the porous media channel 108) can indicate that multiple contact miscibility has not been achieved between the oil composition and the fluid at the test pressure and the test temperature. The absence of an interface can indicate that multiple contact miscibility has been achieved between the oil composition and the fluid at the test pressure and test temperature. For example, FIGS. 4A and 4B show still images captured with an optical investigation sub-system while flowing an aliquot of a fluid through the porous media channel 108 to displace an oil composition, in accordance with the system 300 and method described above. In FIG. 4A, an interface 400 is present between the fluid (which appears black) and the oil composition (which appears white) at the fluid-oil displacement front. This indicates that at the temperature and pressure at which the image was captured, multiple contact miscibility has not been achieved. In contrast, in FIG. 4B, an interface is not present between the fluid (which appears black) and the oil composition (which appears white) at the fluid-oil displacement front. This indicates that at the temperature and pressure at which the image of FIG. 4B was captured, multiple contact miscibility has been achieved.

Instead of or in addition to detecting the presence or absence of an interface between the fluid and the oil composition at the fluid-oil displacement front, the step of conducting an optical investigation can include determining the displacement efficiency of the fluid and the oil composition. For example, after flow of the fluid through the porous media channel 108 is complete, the optical investigation sub-system can be used to determine the oil composition displacement efficiency in at least a section of the porous media channel 108. If the displacement efficiency is at or near 100%, it can be determined that at the temperature and pressure at which the experiment was conducted, multiple contact miscibility has been achieved.

Optionally, the step of conducting an optical investigation can be at least partially automated. For example, as mentioned above, the control system can include image processing and analysis software that can detect the presence or absence of an interface between the fluid and the oil composition at the fluid-oil displacement front and/or calculate the oil composition displacement efficiency in at least a section of the porous media channel 108.

The optical investigation can be carried out in real time (e.g. concurrently with flowing the aliquot of the fluid through the porous media channel 108, or shortly after the flow of fluid has stopped), or can be carried out at a later time (e.g. based on still images or a video recording of the porous media channel 108 during flow of the fluid through the porous media channel 108).

In some examples, the miscibility of the oil composition and the fluid can be assessed in less than about 30 minutes. That is, it can take less than about 30 minutes (e.g. about 10 minutes) from when the first syringe pump 306 is engaged to load the aliquot of the oil composition into the porous media channel 108 until it is determined whether the fluid and the oil composition are miscible at the test pressure and the test temperature.

Optionally, the method can be repeated with subsequent test pressures, such as higher test pressures or lower test pressures. For example, if the optical investigation indicates that the multiple contact miscibility of the fluid and the oil composition has not been achieved at the original test pressure and test temperature, the test can be repeated at a higher test pressure; alternatively, if the optical investigation indicates that multiple contact miscibility has been achieved between the fluid and the oil composition at the original test pressure and test temperature, the test can be repeated at a lower test pressure, to ascertain the multiple contact minimum miscibility pressure. More specifically, in some examples, in order to repeat the experiment, the microfluidic device 100 can first be cleaned (e.g. by flushing the oil composition through the microfluidic device 100). Then, while heating the microfluidic device 100 to the test temperature and while applying back pressure to the porous media channel 108, the porous media channel 108 can be loaded with a subsequent aliquot of the oil composition. Then, while continuing to apply back-pressure to the porous media channel 108 to maintain the porous media channel 108 at a subsequent test pressure (i.e. a pressure that is higher or lower than the original test pressure), a subsequent aliquot of the fluid can be forced to flow through the porous media channel 108, to displace at least some of the subsequent aliquot of the oil composition from the porous media channel 108. Either while the subsequent aliquot of the fluid is flowing through the porous media channel 108, or after flow of the subsequent aliquot of the fluid through the porous media channel 108 has been stopped, an optical investigation of the porous media channel 108 can be conducted, in order to assess the miscibility of the oil composition and the fluid at the subsequent test pressure and the test temperature.

The method can optionally be serially repeated (with further test pressures) until, for example, the multiple contact minimum miscibility pressure of the oil composition and the fluid is ascertained.

Optionally, the method can be repeated with subsequent fluid concentrations, such as a fluid with higher or lower concentration of certain compounds such as ethane, propane, butane, pentane, hexane, heptane, H₂S or CO₂, to ascertain the multiple contact minimum miscibility concentration. For example, if the optical investigation indicates that the multiple contact miscibility of the fluid and the oil composition has not been achieved at the original test pressure, original test temperature, and original concentration of compounds in the fluid (i.e. also referred to as a “first fluid concentration”), the test can be repeated with a subsequent aliquot of fluid having a higher concentration of compounds in the fluid (referred to as a “second fluid concentration”); alternatively, if the optical investigation indicates that multiple contact miscibility has been achieved between the fluid and the oil composition at the original test pressure, original temperature, and original fluid concentration, the test can be repeated at a lower concentration of compounds in the fluid, to ascertain the multiple contact minimum miscibility concentration.

Additional examples of microfluidic devices will now be described with reference to FIGS. 5 to 13. The microfluidic devices of FIGS. 5 to 13 may be used in the system 300 of FIG. 3, or in other systems. The microfluidic devices of FIGS. 5 to 13 may be used according to the methods described above, or according to other methods.

Referring first to FIG. 5, an additional example of a microfluidic device is shown. Features in FIG. 5 that are like those of FIGS. 1 to 2B will be referred to with like reference numerals as in FIGS. 1 to 2B, incremented by 400.

Similarly to the microfluidic device 100 of FIGS. 1 to 2B, the microfluidic device 500 includes a substrate 502 that has a porous media channel 508; an oil inlet port 526 that is in fluid communication with the porous media channel 508 via an oil inlet channel 530, a first feeder channel 534, and a second feeder channel 536; a fluid inlet port 528 that is in fluid communication with the porous media channel 508 via a fluid inlet channel 532, the first feeder channel 534, and the second feeder channel 536; and an outlet port 538 that is in fluid communication with the porous media channel 508 via an outlet channel 540. However, the length of the porous media channel 508 of the microfluidic device 500 is significantly longer than the length of the porous media channel 108 of the microfluidic device 100. Particularly, the length of the porous media channel 508 can be about 6.5 cm. Furthermore, in order to allow for the porous media channel 508 to fit within the microfluidic substrate 502, the porous media channel 508 is serpentine, and winds in a widthwise fashion from one end of the microfluidic substrate 502 to the other. The windings can be spaced apart by a spacing 542 of, for example, about 0.08 cm. As mentioned above, a porous media channel of a relatively long length can enable multiple contact miscibility of an oil composition and a fluid, by increasing the number of contacts between the oil composition and the fluid.

Referring now to FIG. 6, an additional example of a microfluidic device is shown. Features in FIG. 6 that are like those of FIGS. 1 to 2B will be referred to with like reference numerals as in FIGS. 1 to 2B, incremented by 500.

Similarly to the microfluidic device 100 of FIGS. 1 to 2B, the microfluidic device 600 includes a substrate 602 that has a porous media channel 608; an oil inlet port 626 that is in fluid communication with the porous media channel 608 via an oil inlet channel 630, a first feeder channel 634, and a second feeder channel 636; a fluid inlet port 628 that is in fluid communication with the porous media channel 608 via a fluid inlet channel 632, the first feeder channel 634, and the second feeder channel 636; and an outlet port 638 that is in fluid communication with the porous media channel 608 via an outlet channel 640. However, the length of the porous media channel 608 of the microfluidic device 600 is significantly longer than the length of the porous media channel 108 of the microfluidic device 100, and also longer than the length of the porous media channel 508 of the microfluidic device 500 of FIG. 5. Particularly, the length of the porous media channel 608 can be about 24 cm. Furthermore, like the porous media channel 508 of FIG. 5, the porous media channel 608 is serpentine, and winds in a widthwise fashion from one end of the microfluidic substrate 602 to the other. The windings can be spaced apart by a spacing 642 of, for example, about 0.08 cm. As mentioned above, a porous media channel of a relatively long length can enable multiple contact miscibility of an oil composition and a fluid, by increasing the number of contacts between the oil composition and the fluid.

Referring now to FIGS. 7A and 7B, another example of microfluidic device is shown. Features in FIGS. 7A and 7B that are like those of FIGS. 1 to 2B will be referred to with like reference numerals as in FIGS. 1 to 2B, incremented by 600.

Referring first to FIG. 7A, similarly to the microfluidic device 100 of FIGS. 1 to 2B, the microfluidic device 700 includes a substrate 702 that has a porous media channel 708; an oil inlet port 726 that is in fluid communication with the porous media channel 708 via an oil inlet channel 730, a first feeder channel 734, and a second feeder channel 736; a fluid inlet port 728 that is in fluid communication with the porous media channel 708 via a fluid inlet channel 732, the first feeder channel 734, and the second feeder channel 736; and an outlet port 738 that is in fluid communication with the porous media channel 708 via an outlet channel 740. Similarly to the microfluidic devices 500 and 600 of FIGS. 5 and 6, respectively, the porous media channel 708 is relatively long and is serpentine.

Referring still to FIG. 7A, unlike the microfluidic device 100 of FIGS. 1 to 2B, the oil inlet channel 730 and the fluid inlet channel 732 are both relatively long, and are serpentine. As mentioned above, larger lengths can result in a relatively a large pressure drop across the length of the oil inlet channel 730 and the fluid inlet channel 732, respectively. This can help to control the flow of oil compositions and fluids (e.g. fluids with a low viscosity, including gases such as carbon dioxide), and can also help to dampen flow pulsations caused, for example, by temperature fluctuations.

Referring now to 7B, unlike the microfluidic device 100 of FIGS. 1 to 2B, the dividers 710 of the porous media channel 708 are in the form posts that have a square cross-section.

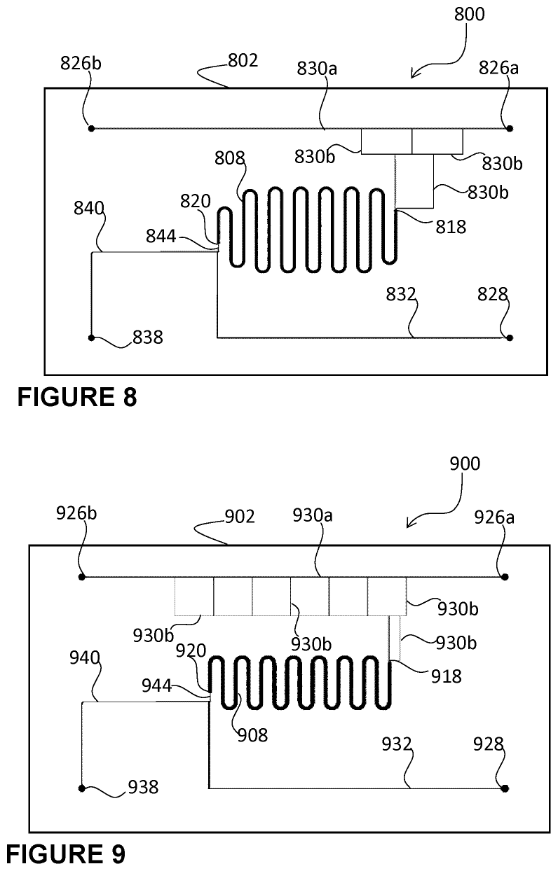

Referring now to FIG. 8, another example of microfluidic device is shown. Features in FIG. 8 that are like those of FIGS. 1 to 2B will be referred to with like reference numerals, incremented by 700.

Similarly to the microfluidic device 100 of FIGS. 1 to 2B, the microfluidic device 800 includes a substrate 802 that has a porous media channel 808. However, the oil inlet channel, the fluid inlet channel, and outlet channel are of a different configuration from those of FIGS. 1 to 2B. Particularly, in the microfluidic device 800, the fluid inlet port 828 and the outlet port 838 are both in fluid communication with the second end 820 of the porous media channels 808. The fluid inlet port 828 is in fluid communication with the second end 820 of the porous media channel 808 via a fluid inlet channel 832 and a junction channel 844, and the outlet port 838 is in fluid communication with second end 820 of the porous media channel 808 via an outlet channel 840 and the junction channel 844.

Furthermore, the microfluidic device 800 includes multiple oil inlet ports, and multiple pathways from the oil inlet ports to the porous media channel 808. More specifically, the microfluidic device 800 includes a first oil inlet port 826a and a second oil inlet port 826b. The first oil inlet port 826a and the second oil inlet port 826b are in fluid communication with each other via a first oil inlet channel 830a, and are in fluid communication with the first end 818 of the porous media channel 808 via the first oil inlet channel 830a and a network of secondary oil inlet channels 830b (only some of which are labelled). The first oil inlet channel 830a extends from the first oil inlet port 826a to the second oil inlet port 826b, and the network of secondary oil inlet channels 830b branches off of the first oil inlet channel 830a and extends towards the porous media channel 808. The secondary oil inlet channels 830b can have a cross-sectional area (also referred to as a “second cross-sectional area”) that is less than the cross-sectional area of the first oil inlet channel 830a (also referred to herein as a “first cross-sectional area”). That is, the secondary oil inlet channels 830b can have a depth (also referred to herein as a “second depth”) that is less than the depth of the first oil inlet channel 830a (also referred to herein as a “first depth”), and/or a width (also referred to herein as a “second width”) that is less than the width of the first oil inlet channel 830a (also referred to herein as a “first width”). In the example shown, the first depth and second depth are both about 0.50 microns, the first width is about 50 microns, and the second width is about 5 microns. Providing the microfluidic chip 800 with multiple oil inlet ports and with multiple pathways (particularly of decreasing cross-sectional area) from the oil inlet ports to the porous media channel 808 can prevent or minimize plugging of the microfluidic device 800. For example, if a relatively large particle in the oil composition were to plug one of the secondary oil inlet channels 830b, the oil composition could continue to flow through the remaining secondary oil inlet channels 830b. That is, the first oil inlet channel 830a and second oil inlet channels 830b provide a “filter zone” for the oil composition entering the microfluidic device 800, and by passing the aliquot of the oil composition through the filter zone prior to loading the aliquot of the oil composition into the porous media channel 808, plugging of the microfluidic device 800 can be prevented or minimized.

Similarly to the microfluidic devices 500 and 600 of FIGS. 5 and 6, respectively, the porous media channel 808 is relatively long and is serpentine. For example, the length of the porous media channel can be about 9.7 cm.

Referring now to FIG. 9, another example of microfluidic device is shown. Features in FIG. 9 that are like those of FIG. 8 will be referred to with like reference numerals, incremented by 100.

The microfluidic device 900 is similar to the microfluidic device 800, and includes a substrate 902 that has a porous media channel 908; a fluid inlet port 928 that is in fluid communication with the second end 920 of the porous media channel 908 via a fluid inlet channel 932 and a junction channel 944; an outlet port 938 in fluid communication with the second end 920 of the porous media channel 908 via an outlet channel 940 and the junction channel 944; a first oil inlet port 926a and a second oil inlet port 926b that are in fluid communication with each other via a first oil inlet channel 930a, and are in fluid communication with the first end 918 of the porous media channel 908 via the first oil inlet channel 930a and a network of secondary oil inlet channels 930b. However, the microfluidic device 900 includes a larger number of secondary oil inlet channels 930b than that of the microfluidic device 800, to further prevent or minimize plugging of the microfluidic device 900. Furthermore, the porous media channel 908 of the microfluidic device 900 is slightly shorter than that of FIG. 8 (i.e. the length of the porous media channel 908 is about 6.5 cm).

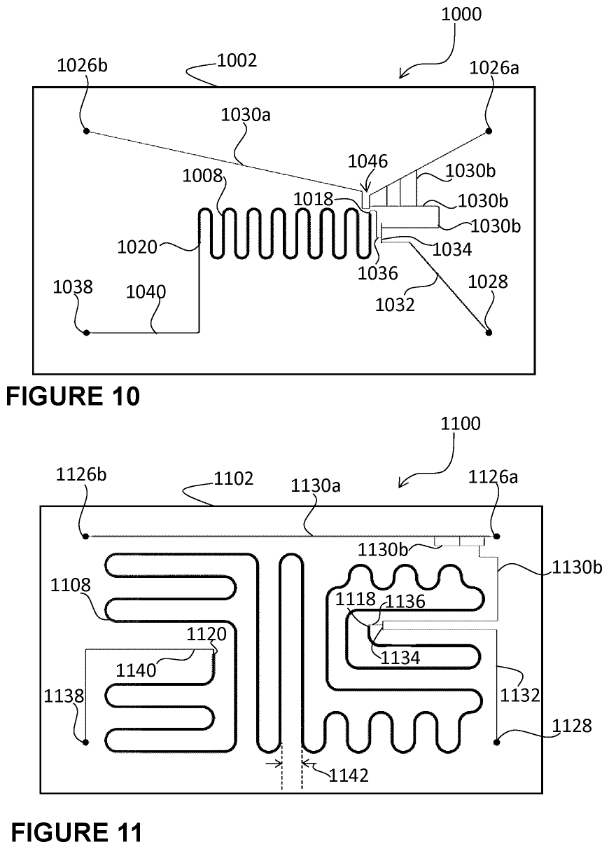

Referring now to FIG. 10, another example of microfluidic device is shown. Features in FIG. 10 that are like those of FIG. 8 will be referred to with like reference numerals, incremented by 200.

The microfluidic device 1000 is similar to the microfluidic device 800, and includes a substrate 1002 that has a porous media channel 1008; a fluid inlet port 1028 that is in fluid communication with the porous media channel 1008 via fluid inlet channel 1032; an outlet port 1038 that is in fluid communication with the second end 1020 of the porous media channel 1008 via an outlet channel 1040; and a first oil inlet port 1026a and a second oil inlet port 1026b that are in fluid communication with each other via a first oil inlet channel 1030a, and are in fluid communication with the first end 1018 of the porous media channel 1008 via the first oil inlet channel 1030a and a network of secondary oil inlet channels 1030b, which act as a filter zone.

However, unlike the microfluidic device 800, the fluid inlet port 1028 is in fluid communication with the first end 1018 of the porous media channel 1008, via a first feeder 1034 channel and a second feeder channel 1036 (similar to the feeder channels of FIGS. 1 to 2B). Furthermore, the network of secondary oil inlet channels 1030b is in fluid communication with the porous media channel 1008 via the first feeder channel 1034 and the second feeder channel 1036. Furthermore, the microfluidic device 1000 is configured for operation below the saturation pressure of an oil composition. That is, the microfluidic device 1000 is configured to allow for an oil composition to flash into a gas phase and a liquid phase within the microfluidic device 1000 and upstream of the porous media channel 1008, so that in use, a gas phase and a liquid phase are loaded into the porous media channel 1008. Particularly, the network of secondary oil inlet channels 1030b has a reduced cross-sectional area as compared to the cross-sectional area of the first feeder channel 1034 and the second feeder channel 1036. For example, the depth of the network of secondary oil inlet channels 1030b (i.e. the second depth) can be about 1 micron, and the depth of the first feeder channel 1034 and the second feeder channel 1036 can be about 50 microns. By providing the network of secondary oil inlet channels 1030b with a reduced depth, a pressure drop is induced across the length of the secondary oil inlet channels 1030b. In use, if an aliquot of a saturated oil composition is loaded into the microfluidic device 1000 while maintaining the porous media channel 1008 below the saturation pressure of the oil composition, flashing of the aliquot of the oil composition into a gas phase and a liquid phase will occur downstream of the network of secondary oil inlet channels 1030b and upstream of the porous media channel 1008, i.e. in the first feeder channel 1034 and/or the second feeder channel 1036. The area in which flashing occurs can be referred to herein as a “flash zone” of the microfluidic device 1000. This gas phase and liquid phase mixture can then be loaded into the porous media channel 1008.

Referring still to FIG. 10, in the example shown, the first oil inlet channel 1030a is non-linear, and is shaped to include a portion 1046 that is proximate the porous media channel 1008. This can allow for the portion 1046 to be within the viewing window of the optical investigation system.

Referring now to FIG. 11, another example of a microfluidic device is shown. Features in FIG. 11 that are like those of FIG. 10 will be referred to with like reference numerals, incremented by 100.

Similarly to the microfluidic device 1000 of FIG. 10, the microfluidic device 1100 includes a substrate 1102 that has a porous media channel 1108; a fluid inlet port 1128 that is in fluid communication with the first end 1118 of the porous media channel 1108 via fluid inlet channel 1132, a first feeder channel 1134, and a second feeder channel 1136; a first oil inlet port 1126a and a second oil inlet port 1126b that are in fluid communication with each other via a first oil inlet channel 1130a, and are in fluid communication with the first end 1118 of the porous media channel 1108 via the first oil inlet channel 1130a and a network of secondary oil inlet channels 1130b, which act as a filter zone; and an outlet port 1138 that is in fluid communication with the second end 1120 of the porous media channel 1108 via an outlet channel 1140.

Referring still to FIG. 11, the porous media channel 1108 has a porous media channel length that is relatively large—i.e. about 31 cm. Furthermore, the porous media channel 1108 is serpentine, however it is wound both widthwise and lengthwise along the microfluidic substrate 1102. Furthermore, the windings of the porous media channel 1108 are spaced further apart than in the previous figures. For example, while the windings in FIG. 5 are spaced apart by a spacing 542 about 0.08 cm, the windings in FIG. 11 may be spaced apart by a spacing 1142 about 0.16 cm. Spacing apart the windings may help to distribute stresses evenly on the surface of the substrate 1102, which may allow the microfluidic device 1100 to operate under high pressures.

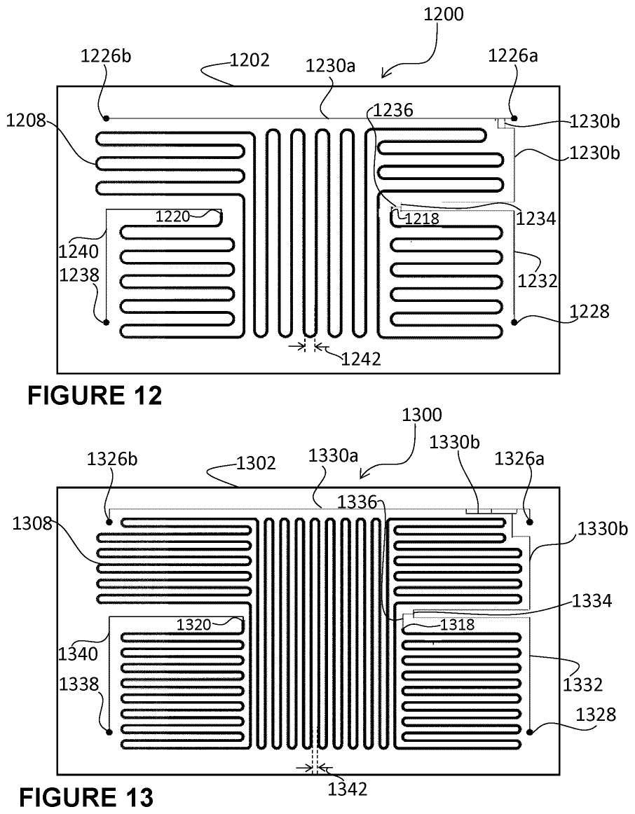

Referring now to FIG. 12, another example of a microfluidic device is shown. Features in FIG. 12 that are like those of FIG. 11 will be referred to with like reference numerals, incremented by 100.

Similarly to the microfluidic device 1100 of FIG. 11, the microfluidic device 1200 includes a substrate 1202 that has a porous media channel 1208; a fluid inlet port 1228 that is in fluid communication with the first end 1218 of the porous media channel 1208 via fluid inlet channel 1232, a first feeder channel 1234, and a second feeder channel 1236; a first oil inlet port 1226a and a second oil inlet port 1226b that are in fluid communication with each other via a first oil inlet channel 1230a, and are in fluid communication with the first end 1218 of the porous media channel 1208 via the first oil inlet channel 1230a and a network of secondary oil inlet channels 1230b (which act as a filter zone), and via the first feeder channel 1234 and the second feeder channel 1236; and an outlet port 1238 that is in fluid communication with the second end 1220 of the porous media channel 1208 via an outlet channel 1240.

The porous media channel 1208 has a porous media channel length that is longer than that of FIG. 11—i.e. about 54 cm. Similarly to the porous media channel 1108 of FIG. 11, the porous media channel 1208 is serpentine and is wound both widthwise and lengthwise along the microfluidic substrate 1202. Furthermore, the windings of the porous media channel are spaced apart by a spacing 1242 of about 0.08 cm.

Referring now to FIG. 13, another example of a microfluidic device is shown. Features in FIG. 13 that are like those of FIG. 11 will be referred to with like reference numerals, incremented by 200.

Similarly to the microfluidic device 1100 of FIG. 11, the microfluidic device 1300 includes a substrate 1302 that has a porous media channel 1308; a fluid inlet port 1328 that is in fluid communication with the first end 1318 of the porous media channel 1308 via fluid inlet channel 1332, a first feeder channel 1334, and a second feeder channel 1336; a first oil inlet port 1326a and a second oil inlet port 1326b that are in fluid communication with each other via a first oil inlet channel 1330a, and are in fluid communication with the first end 1318 of the porous media channel 1308 via the first oil inlet channel 1330a and a network of secondary oil inlet channels 1330b (which act as a filter zone), and via the first feeder channel 1334 and the second feeder channel 1336; and an outlet port 1338 that is in fluid communication with the second end 1320 of the porous media channel 1308 via an outlet channel 1340.

The porous media channel 1308 has a porous media channel length that is longer than that of FIG. 11—i.e. about 92 cm. Similarly to the porous media channel 1108 of FIG. 11, the porous media channel 1308 is serpentine and is wound both widthwise and lengthwise along the microfluidic substrate 1302. Furthermore, the windings of the porous media channel are spaced apart by a spacing 1342 of about 0.04 cm.

As used herein, the term “about” indicates a degree of variability in a value or range, for example, within 10%, within 5%, or within 1% of a stated value or of a stated limit of a range.

All numerical ranges listed herein are inclusive of the bounds of those ranges. For example, the statement that a certain measurement may be “between 25 cm and about 75 cm” means that the measurement may be 25 cm, or 75 cm, or any number therebetween.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

### EXAMPLES

The multiple contact minimum miscibility pressure of carbon dioxide and three oil compositions (“Pennsylvania oil”, dodecane, and pentane (43 mol %) and hexadecane mixture) was assessed using the microfluidic device of FIG. 5 and the system of FIG. 3, according to the methods described herein.

The test results were compared to the MMP reported in literature for various other devices, systems and techniques (e.g. MRI). Results are shown in table 1.

| | | Test Results | Literature Data | |
|---|---|---|---|---|
| | T | Multiple Contact | Multiple Contact | |
| Sample | (° C.) | MMP (bar) | MMP (bar) | Ref |
| Pennsylvania Dead Oil | 25 | 58 | 55.2 ± 0.7 | 1 |
| Dodecane | 30 | 70 | 70.8 | 2 |
| | 37.8 | 82.5 | 82.6 | 2 |
| Pentane (43 mol %) + | 50 | 97.5 | 103.4; 107 | 3, 4 |
| Hexadecane | 53 | 105 | 116 | 3 |
| | 71.7 | 142.5 | 151.5 | 3 |

The results in table 1 indicate that the systems, methods, and devices herein can provide results that are comparable to other methods.

### REFERENCES FOR DATA IN TABLE 1

1. Nguyen, Phong, et al. “Fast fluorescence-based microfluidic method for measuring minimum miscibility pressure of CO2 in crude oils.” Analytical chemistry 87.6 (2015): 3160-3164.
2. Liu, Yu, et al. “Estimation of minimum miscibility pressure (MMP) of CO2 and liquid n-alkane systems using an improved MRI technique.” Magnetic resonance imaging 34.2 (2016): 97-104.
3. Christiansen, Richard L., and Hiemi Kim Haines. “Rapid measurement of minimum miscibility pressure with the rising-bubble apparatus.” SPE Reservoir Engineering 2.04 (1987): 523-527.
4. Elsharkawy, Adel M., Fred H. Poettmann, and Richard L. Christiansen. “Measuring CO2 minimum miscibility pressures: slim-tube or rising-bubble method?.” Energy & fuels 10.2 (1996): 443-449.

1. A method for assessing miscibility of an oil composition and a fluid, the method comprising:
a. in a microfluidic device, heating or cooling a porous media channel to a test temperature
b. while applying back-pressure to the porous media channel, loading the porous media channel with an aliquot of the oil composition;
c. while applying back-pressure to the porous media channel to maintain the porous media channel at a test pressure, flowing an aliquot of the fluid through the porous media channel to displace at least some of the aliquot of the oil composition from the porous media channel; and
d. during and/or after step c., conducting an optical investigation of the porous media channel to assess the miscibility of the oil composition and the fluid at the test pressure and test temperature.

2. (canceled)

3. (canceled)

4. (canceled)

5. (canceled)

6. (canceled)

7. (canceled)

8. (canceled)

9. (canceled)

10. (canceled)

11. The method of claim 1, wherein the porous media channel has a porous media channel length of between about 25 cm and about 75 cm and a porous media channel width of between about 5 microns and about 500 microns.

12. The method of claim 1, wherein the porous media channel comprises a network of fluid pathways, and each fluid pathway has a fluid pathway width of between about 1 micron and about 50 microns.

13. (canceled)

14. (canceled)

15. The method of claim 1, wherein:
step c. comprises flowing the aliquot of the fluid into the porous media channel via a fluid inlet channel;
the porous media channel has a porous media channel cross-sectional area; and
the fluid inlet channel has a fluid inlet channel cross-sectional area that is less than the porous media channel cross-sectional area.

16. The method of claim 1, further comprising, prior to loading the porous media channel with the aliquot of the oil composition, flashing the aliquot of the oil composition into a liquid phase and a gas phase in a flash zone of the microfluidic device.

17. (canceled)

18. The method of claim 1, further comprising passing the aliquot of the oil composition through a filter zone of the microfluidic device to filter the aliquot of the oil composition prior to loading the aliquot of the oil composition into the porous media channel.

19. (canceled)

20. A microfluidic system comprising:
a microfluidic device comprising a microfluidic substrate, the microfluidic substrate comprising a porous media channel, an oil inlet port in fluid communication with the porous media channel, a fluid inlet port in fluid communication with the porous media channel, and an outlet port in fluid communication with the porous media channel, wherein the porous media channel comprises a plurality of dividers that provide the porous media channel with a network of fluid pathways;
an oil injection sub-system in fluid communication with the oil inlet port for forcing an oil composition into the network of fluid pathways;
a fluid injection sub-system in fluid communication with the fluid inlet port for forcing a fluid through the network of fluid pathways from the fluid inlet port towards the outlet port;
a pressure regulation sub-system for regulating the pressure in the network of fluid pathways;
a manifold providing fluid communication between the microfluidic substrate and the oil injection sub-system, the fluid injection sub-system, and the pressure regulation sub-system;
a temperature regulation sub-system for regulating the temperature of at least the microfluidic device; and
an optical investigation sub-system for optically accessing at least a portion of the porous media channel.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. A microfluidic device comprising:
a microfluidic substrate having a porous media channel, an oil inlet port in fluid communication with the porous media channel, a fluid inlet port in fluid communication with the porous media channel, and an outlet port in fluid communication with the porous media channel;
wherein the porous media channel comprises a plurality of dividers that provide the porous media channel with a network of fluid pathways.

37. (canceled)

38. (canceled)

39. (canceled)

40. (canceled)

41. (canceled)

42. The microfluidic device of claim 36, wherein the porous media channel has a porous media channel length of between about 25 cm and about 75 cm.

43. (canceled)

44. (canceled)

45. The microfluidic device of claim 36, wherein the porous media channel has a porous media channel width of between about 50 microns and 300 microns.

46. (canceled)

47. (canceled)

48. The microfluidic device of claim 36, wherein the fluid pathways have a pathway width of between about 2 microns and about 20 microns.

49. The microfluidic device of claim 36, wherein the dividers are in the form of posts that are created by etching fluid pathways into the substrate.

50. The microfluidic device of claim 49, wherein the posts are positioned in an array.

51. The microfluidic device of claim 49, wherein the posts are positioned randomly.

52. The microfluidic device of claim 36, further comprising an oil inlet channel extending towards the porous media channel from the oil inlet port, an outlet channel extending towards the porous media channel from the outlet port, and a fluid inlet channel extending towards the porous media channel from the fluid inlet port.

53. The microfluidic device of claim 52, further comprising at least a first feeder channel, wherein the oil inlet channel is in fluid communication with the porous media channel via the first feeder channel, wherein the oil inlet channel has an oil inlet channel cross-sectional area, and wherein the first feeder channel has a first feeder channel cross-sectional area that is greater than the oil inlet channel cross-sectional area.

54. (canceled)

55. The microfluidic device of claim 52, wherein
the porous media channel has a porous media channel depth;
the fluid inlet channel has a fluid inlet channel depth; and
the fluid inlet channel depth is less than the porous media channel depth.

56. (canceled)

57. The microfluidic device of claim 55, wherein the fluid inlet channel depth is between about 25 times less and 75 times less than the porous media channel depth.

58. The microfluidic device of claim 36, further comprising a secondary oil inlet port, wherein the oil inlet port and the secondary oil inlet port are in fluid communication with each other via a first oil inlet channel.

59. The microfluidic device of claim 58, further comprising a network of secondary oil inlet channels, wherein the first oil inlet channel is in fluid communication with the porous media channel via the network of secondary oil inlet channels, wherein the first oil inlet channel has a first cross-sectional area, wherein the secondary oil inlet channels each have a second cross-sectional area, and wherein the second cross-sectional area is less than the first cross-sectional area to form a filter zone in the microfluidic substrate.

60. (canceled)
